(19) United States
(12) Patent Application Publication
(10) Pub. No.: US 2026/0345903 A1
(43) Pub. Date: Nov. 5, 2026

(54) SYSTEM AND METHOD FOR AUTOMATICALLY PRIMING AN INFUSION LINE

(71) Applicant: CareFusion 303, Inc., San Diego, CA (US)

(72) Inventor: Chun Keat OOI, Limerick (IE)

(21) Appl. No.: 19/123,714

(22) PCT Filed: Oct. 28, 2022
(86) PCT No.: PCT/US22/48295

§ 371 Date: Apr. 23, 2025

(51) Int. Cl. A61M 5/172; A61M 5/14; A61M 5/142

(52) U.S. Cl. CPC A61M 5/172; A61M 5/142; A61M 2005/1402; A61M 2205/3327; A61M 2205/3331; A61M 2205/502

(57) ABSTRACT

The subject technology includes a pressure priming apparatus configured such that, responsive to fluid flow into the pressure priming apparatus, a fluid input to the pressure priming apparatus has a first fluid breakthrough backpressure, and a fluid output of the pressure priming apparatus has a second fluid breakthrough backpressure greater than the first fluid breakthrough backpressure. A pump controller is configured to determine a priming pressure threshold greater than the first fluid breakthrough backpressure and less than the second fluid breakthrough backpressure, and initiate a priming of a fluid tubing connected to the fluid input of the pressure priming apparatus with a fluid. The pump controller automatically terminates the priming of the fluid tubing when the priming pressure of the fluid satisfies the determined priming pressure threshold, and provides an indication that the fluid tubing is primed responsive to the fluid satisfying the determined priming pressure threshold.

### BACKGROUND

There are a variety of situations where fluid is infused to a patient. Applications of fluid delivery systems include (but are not limited to) intravenous (IV) infusion, intra-arterial infusion, infusion of enteral solutions, infusion of medication to the epidural space, and diagnostic infusion to determine vascular characteristics of the arterial, urinary, lymphatic, or cerebrospinal systems.

Fluid delivery systems for infusing fluid to a patient typically include a supply of the fluid to be administered, an infusion needle or cannula, an administration set connecting the fluid supply to the cannula, and a flow control device, such as a positive displacement infusion pump. The administration set typically comprises a length of flexible tubing. The cannula is mounted at the distal end of the flexible tubing for insertion into a patient's blood vessel or other body location to deliver the fluid infusate to the patient.

Some IV sets for use with an infusion pump include an anti-siphon valve at the distal end to prevent unintended free-flow and back-flow. This also prevents priming the set by gravity. Instead, priming is achieved by manually removing a sterile cap from a luer and pressing a prime button on pump. The priming mechanism pushes a pre-set volume of fluid through the tubing (typically exceeding air/dead space of the IV set). In many situations, the clinician has to manually monitor the priming process and stop the pump once fluid starts coming out the distal end. It can be a troublesome/messy/time-consuming process in requiring another receptable to hold the excess fluid and/or risky in terms of sterility, hazardous/controlled medication, etc. All these add to clinician workload, inefficiencies in workflow and potential clinical risk.

### SUMMARY

According to various aspects of the subject technology, a system for automatically priming an infusion line is provided. The system comprises a pressure priming apparatus configured such that, responsive to fluid flow into the pressure priming apparatus, a fluid input to the pressure priming apparatus has a first fluid breakthrough backpressure, and a fluid output of the pressure priming apparatus has a second fluid breakthrough backpressure greater than the first fluid breakthrough backpressure; and a pump controller configured to: determine a priming pressure threshold greater than the first fluid breakthrough backpressure and less than the second fluid breakthrough backpressure; initiate a priming of a fluid tubing connected to the fluid input of the pressure priming apparatus with a fluid; automatically terminate the priming of the fluid tubing when the priming pressure of the fluid satisfies the determined priming pressure threshold; and provide an indication that the fluid tubing is primed responsive to the fluid satisfying the determined priming pressure threshold. Other aspects include corresponding apparatuses, methods and processes for implementation of the corresponding system.

A method for automatically priming an infusion line comprises: providing a pressure priming apparatus configured such that, responsive to fluid flow into the pressure priming apparatus, a fluid input to the pressure priming apparatus has a first fluid breakthrough backpressure, and a fluid output of the pressure priming apparatus has a second fluid breakthrough backpressure greater than the first fluid breakthrough backpressure; determine a priming pressure threshold greater than the first fluid breakthrough backpressure and less than the second fluid breakthrough backpressure; initiating a priming of a fluid tubing connected to the fluid input of the pressure priming apparatus with a fluid; automatically terminating the priming of the fluid tubing when the priming pressure of the fluid satisfies the determined priming pressure threshold; and providing an indication that the fluid tubing is primed responsive to the fluid satisfying the determined priming pressure threshold. Other aspects include corresponding apparatuses, systems and processes for implementation of the corresponding method.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 illustrates a caregiver administering an infusion to a patient in a typical healthcare environment.

FIG. 2A depicts an example infusion pump set-up, shown in use in its intended environment, according to various aspects of the subject technology.

FIG. 2B depicts a perspective view of an example infusion device showing an infusion set in place within the infusion device, according to various aspects of the subject technology.

FIGS. 3A and 3B depict an exploded illustration of an example pressure priming apparatus, according to various aspects of the subject technology.

FIGS. 4A, 4B, and 4C depict an example of priming an infusion line using the pressure priming system of the subject technology.

FIG. 5 depicts an example process for automatically priming an infusion line, according to various implementations of the subject technology.

FIG. 6 is a conceptual diagram illustrating an example electronic system for automatically priming an infusion line, according to aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology provides a system for automatically priming an infusion line. According to various implementations, a pressure priming apparatus includes a first portion and a second portion that are removably coupled together. When the pressure priming apparatus is connected to a fluid tubing, the pressure priming apparatus enforces a directional flow of fluid flowing through the tubing, as well as controlling a flow of the fluid into a chamber of the priming apparatus by constraining the fluid using two backpressure regulators at the input and output of the apparatus. In this regard, different backpressures are generated at the input and at the output, with the backpressure at the output being greater than at the input of the pressure apparatus. Accordingly, a pump controller initiates a priming of the fluid tubing by pumping fluid through the tubing and into the chamber of the priming apparatus until the pressure within the tubing is greater than the backpressure of the input but less than the backpressure of the output. For example, the pump system may preselect a threshold backpressure value between the input and output backpressures, and determine the tubing to be primed when the interior fluidic pressure within the tubing reaches the threshold backpressure.

As used herein, a “fluid breakthrough backpressure” is a minimum required pressure at which the fluid flowing through the tubing must flow in order to flow through the tubing (overcoming the breakthrough backpressure). As will be described further, the first portion of the pressure priming apparatus may further include a one-way valve at the input to control the directional flow of the fluid. The valve may generate a first breakthrough backpressure as a function of the valve's cracking pressure (including part tolerances). The second portion of the pressure priming apparatus may include a hydrophobic filter (e.g., a hydrophobic membrane) at its output, which generates a second breakthrough backpressure based on the hydrophobic filter's water pressure resistance (e.g., liquid entry pressure, including tolerances).

The hydrophobic filter allows air to flow within the tubing and through the filter, but not fluid, eliminating the problems encountered in existing priming techniques. During the priming function, the pump monitors downstream line pressure and once the threshold backpressure is satisfied (e.g., a pressure between the first and second breakthrough backpressures), the pump automatically stops the priming action and the user is notified via a display that the priming is complete. According to various implementations, the threshold backpressure is selected to be higher than the first breakthrough backpressure and to be lower than the second breakthrough backpressure. Because the pressure is greater than the first breakthrough backpressure, the system ensures that the infusion tubing is primed and a manual top-off process is avoided, and because the pressure is lower than the second breakthrough backpressure, the system eliminates or minimizes fluid spill over.

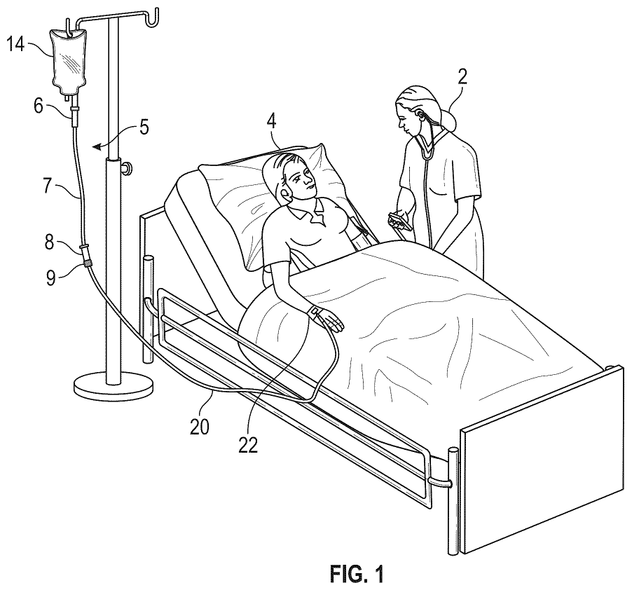

FIG. 1 illustrates a caregiver 2 administering an infusion to a patient 4 in a typical healthcare environment. The patient is receiving infusion fluid through and an IV administration set 5 coupled to the patient's vascular system. The IV administration set 5 may include a drip chamber 6 coupled to the infusion fluid container 14, tubing of an IV line 7 with a proximal end coupled to the drip chamber 6, and a distal end of the IV line 7 coupled to a catheter 22. A roller clamp 8 may be coupled to a length of the IV line 7, between the fluid container 14 and the catheter 22. In some implementations, before the downstream portion 20 of the IV line 7 and the catheter 22 is connected to the fluid container 14, the IV line 7 may terminate at a luer fitting 9 having a priming cap (not shown). In some implementations, the IV priming cap may engage or couple with a portion of the IV line 7, such as the roller clamp 8.

The IV administration set can be gravity primed with the distal end of the IV line 7 coupled to the IV priming cap, or the IV administration set can be primed by separating the fluid connector from the IV priming cap. The priming cap may include a hydrophobic membrane to stop fluid flow through the IV line while allowing air to flow through, to prime the IV administration set. Accordingly, free flow of the fluid through the IV line to the membrane is unimpeded. The clinician ensures that there is a head height difference between the end of the line 7 and the fluid container 14, and gravity fills up the line. The priming cap with the hydrophobic membrane allows air to pass through but not liquid.

The fluid moves freely through the line and luer fitting 9, with gravity causing the fluid to push air through the hydrophobic membrane of the cap on the fitting. The fluid, not having enough force to move through the membrane, stops at the membrane. The clinician may then allow the fluid to settle in the line while attending to other tasks. The clinician may then return to the patient and remove the priming cap. The clinician may then connect the downstream portion 20 of the line and the infusion set is ready to use. In some implementations, the clinician may use a pump to prime the line. However, the pump may push fluid through the membrane, causing fluid spill which may be undesirable, particularly when the fluid includes certain medications (e.g., chemotherapy medications).

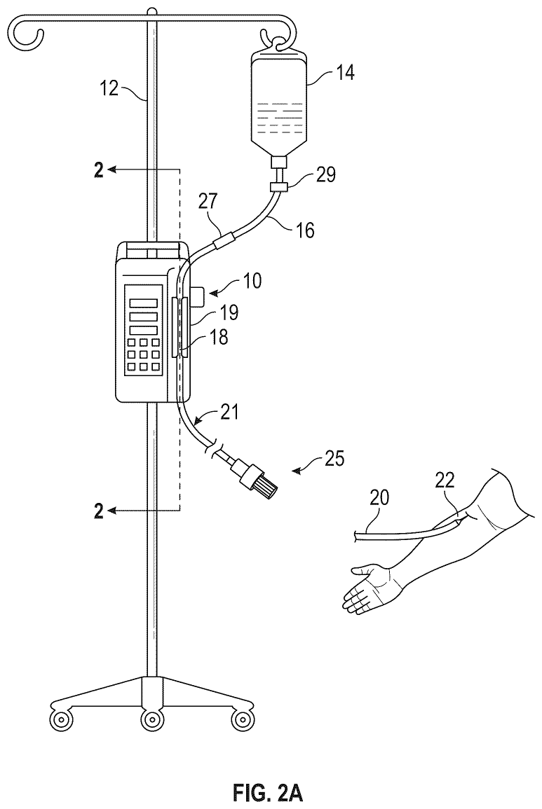

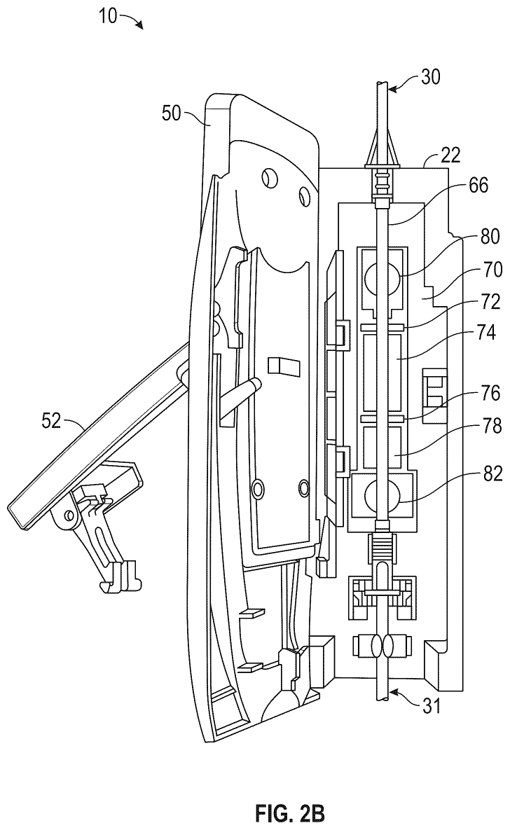

FIG. 2A depicts an example infusion pump set-up 10, shown in use in its intended environment, according to various aspects of the subject technology. In particular, the infusion pump set-up 10 is shown mounted to an intravenous (IV) pole 12 on which a fluid source 14 containing an IV fluid is held. The fluid source 14 is connected in fluid communication with an upstream fluid line 16. The fluid line 16 is a conventional IV infusion type tube typically used in a hospital or medical environment, and is made of any type of flexible tubing appropriate for use to infuse therapeutic fluids into a patient, such as polyvinylchloride (PVC). A flexible pumping fluid line 18 is mounted in operative engagement with a peristaltic pumping apparatus 19, for propelling fluid through a downstream fluid line 20, for example, to a patient's arm. A cannula 22 is mounted at the distal end of the flexible IV tubing 21 for insertion into a patient's blood vessel or other body location to deliver the fluid to the patient.

It will be understood by those skilled in the art that the upstream fluid line 16, the flexible line 18, and the downstream fluid line 20 may be portions of a continuous length of flexible tubing, with the portions defined by the location of the peristaltic pump 19. For convenience, the continuous length of flexible tubing is indicated by numeral 21. A roller clamp (e.g., configured to provide for mechanical compression of the line to block the flow) may be positioned on the downstream fluid line 20 between the pump 10 and the patient's arm 22. In this context, the term “upstream” refers to that portion of the flexible tubing that extends between the fluid source and peristaltic pump, and the term “downstream” refers to that portion of the flexible tubing that extends from the peristaltic pump to the patient.

A pressure priming apparatus 25 may be fitted at an end of infusion line 21, prior to connection of the downstream fluid line 20 to the flexible infusion set 18, and to medical container 14 above. As will be described further, the pressure priming apparatus 25 enforces a directional flow of fluid flowing through the tubing and into a chamber of the priming apparatus, as well as constraining fluid flowing into the apparatus 25 from the tubing 21 using two backpressure regulators. In this manner, that the tubing 21 connected to the tubing may be accurately primed with fluid that is controlled by the pump, without leakage of the fluid during the priming process.

FIG. 2B depicts a perspective view of an example infusion device 10 showing an infusion set in place within the infusion device, according to various aspects of the subject technology. An infusion system for parenteral infusion of a medical fluid to a patient comprises a pump unit, a major part of which comprises a housing which accommodates a cam system (not shown) controlling a plurality of fingers of a pumping mechanism, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls, all in manner known per se.

As shown in FIG. 2B, the infusion pump 10 is shown in perspective view with the front door 50 open, showing the upstream fluid line portion 30 (e.g., a portion of upstream infusion line 16) and downstream fluid line 31 (e.g., downstream portion of infusion line 21) in operative engagement with the pump 10. The infusion pump 10 directly acts on a tube 66 that connects the upstream fluid line 30 to the downstream fluid line 31 to form a continuous fluid conduit, extending from a respective fluid supply to a patient, through which fluid is acted upon by the pump to move fluid downstream to the patient. Specifically, a pumping mechanism 70 acts as the flow control device of the pump to move fluid though the conduit. The depicted references 30, 31, 66 may be used to describe herein portions of one continuous fluid line 21 or, in some implementations, may individually describe portions that are fluidly connected together to form a continuous fluid line. The upstream and downstream fluid lines and/or tube 30, 31, 66 may also be coupled to a pump cassette or cartridge that is configured to be coupled to the pump 10, such as the type described in co-pending U.S. Pat. No. 10,226,571, which is incorporated by reference herein.

The type of pumping mechanism may vary and may be for example, a multiple finger pumping mechanism. For example, the pumping mechanism may be of the “four finger” type and includes an upstream occluding element or finger 72, a primary pumping element or finger 74, a downstream occluding element or finger 76, and a secondary pumping element or finger 78. The “four finger” pumping mechanism and mechanisms used in other linear peristaltic pumps operate by sequentially pressing on a segment of the fluid conduit by means of the cam-following pumping elements (e.g., pumping fingers and valve fingers) 72, 74, 76, and 78, which make four finger pump assembly. The pressure is applied in sequential locations of the conduit, beginning at the upstream end of the pumping mechanism, and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

An upstream pressure sensor 80 may also be included in the pump 10. The upstream pressure sensor is assigned to the flow control device or pumping mechanism 70 and, in this example, is further provided as an integral part of the pump 10. It is mounted to the flow control device 70 and is located adjacent and upstream in relation to the flow control device. The upstream pressure sensor is located upstream from the flow control device, that is, at a location between a fluid supply and the flow control device, so that the connection of the correct fluid supply with the correct pump may be verified before any fluid is pumped to the patient.

A downstream pressure sensor 82 is also included in the pump 10 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient and the flow control device, so that a pressure of a fluid supply to the downstream infusion line 31, and to pressure priming apparatus 25, when connected, may be determined to verify priming of the fluid before the fluid is pumped to the patient.

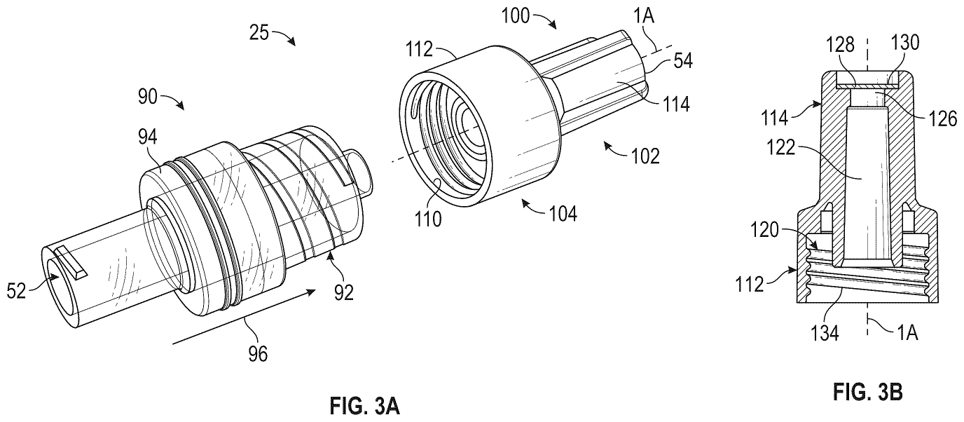

FIG. 3A depicts an exploded illustration of an example pressure priming apparatus, according to various aspects of the subject technology. The pressure priming apparatus 25 of the subject technology includes a fluid input 52 configured to have a first fluid breakthrough backpressure, and a fluid output 54 configured to have a second fluid breakthrough backpressure. A fluid breakthrough backpressure is a fluidic pressure at which a fluid, traveling through an IV tubing coupled to the pressure priming apparatus, must reach before the fluid will flow through the respective input or output of the pressure priming apparatus. While the depicted example is shown as two separate pieces, in some implementations, the pressure priming apparatus may be a single device.

According to various implementations, the pressure priming apparatus 25 includes two portions: a first portion 90, and a second portion 100 which may be removably coupled to the first portion. In the depicted example, the first portion includes a luer fitting and the second portion is a priming cap with its output sealed with a hydrophobic membrane (e.g., embedded therein). The depicted first portion 90 is configured to receive and fluidically seal to the second portion 100. For example, the first portion 90 may include a threaded shaft 92 configured to engage and thread into a threaded receiving cavity 110 of the second portion 100 of the pressure priming apparatus 25. In this regard, the threaded priming cap 100 may be removed from the first portion 90 and disposed of (e.g., thrown away) when priming is completed, and replaced with a downstream portion 20 of an infusion set 21 by way of coupling a threaded connector at an input of the set to the threaded shaft 92. While a threaded luer-type connector is illustrated, it is understood that the two portions may couple and fluidically seal using other connecting mechanisms.

In some implementations, the first portion 90 includes a one-way check valve 94 that restricts the flow of the fluid to one direction 96. The check valve may further generate the first fluid breakthrough backpressure such that fluid will only flow in the indicated direction 96 when a pressure of the fluid satisfies the first fluid breakthrough backpressure (e.g., is greater or equal to 30 kPa±a tolerance). In some implementations, first portion 90 may include an anti-siphon valve with these features.

The IV priming cap 100 includes a body 102 having a proximal segment 112 and a distal segment 114. An inner cavity 110 of the proximal segment 112 defines extends into the body 102 at a proximal end 104, and which is configured to receive a fluid connector therein. The cavity may extend from the proximal end 104 toward the distal end 106 of the body 102. A longitudinal connector cavity axis 1A is shown extending between the proximal and distal ends of the body and, when the first and second portions are connected, may extend the entire length of the pressure priming apparatus 25. The cavity 110 may include helical ridge 134 shaped as a thread along the inner surface of the cavity at proximal end 112. The thread can extend away from the inner surface or into the inner surface 110 of the cover body. The thread can have a thread pitch configured to mesh with a thread of a fluid connector coupled to IV priming cap. The ridge 134 can be formed as any of a helical ridge and a longitudinal ridge. In some implementations, the ridge 134 can be any shape configured to engage with a fluid connector. In this manner, the second portion 100 may fluidically seal to first portion 90 (e.g., threaded shaft 92).

With reference to FIG. 3B, the second portion 100 may include a priming passage 122 therein that extends the length of the portion 100, from cavity 110 to terminate at the fluid output of the distal end 54. At the distal end 54, the second portion 100 may include a filter 128 embedded therein and/or sealing the fluid output. In some implementations, the second portion 100 may include a recess 130 to receive the filter 128 therein. The recess 130 may extend into the distal end of the cover body 102 and a distal portion 126 of the priming passage 122 intersects the recess 130. In some embodiments, the IV priming cap does not include a filter. According to various implementations, the filter 128 is a hydrophobic filter (or hydrophobic membrane). In some examples, the filter 128 comprises polyethylene material, or comprises a membrane coated with polyethylene. The filter 128 may be configured to permit a gas to move through the filter while resisting movement of a liquid through the filter. For example, the filter 128 can be positioned within the priming passage 122, 126, or coupled to a tip portion of the cover body 102.

The hydrophobic filter (and/or priming passage 122, 126) may be configured such that there is little resistance by the filter to air when the filter is not wetted (e.g., less than or equal to 1 kPa±a tolerance) while, when wetted, the resistance increases (e.g., greater than or equal to 60 kPa±a tolerance). According to various implementations, the second fluid breakthrough backpressure produced by the second portion 100 is greater than the first fluid breakthrough backpressure produced by the first portion 90.

Although the present disclosure describes implementations of coupling the second portion 100 with a male luer-type fluid connector 70, it should be appreciated that the IV priming cap can be coupled with other fluid connectors. However, for clarity and brevity, the present disclosure may primarily refer to a male luer fluid connector. Additionally, although the present disclosure describes the pressure priming apparatus 25 relative to an IV administration set, it should be appreciated that the pressure priming apparatus 25 can be coupled with other IV fluid delivery devices and systems. However, for clarity and brevity, the present disclosure will primarily refer to an IV administration set.

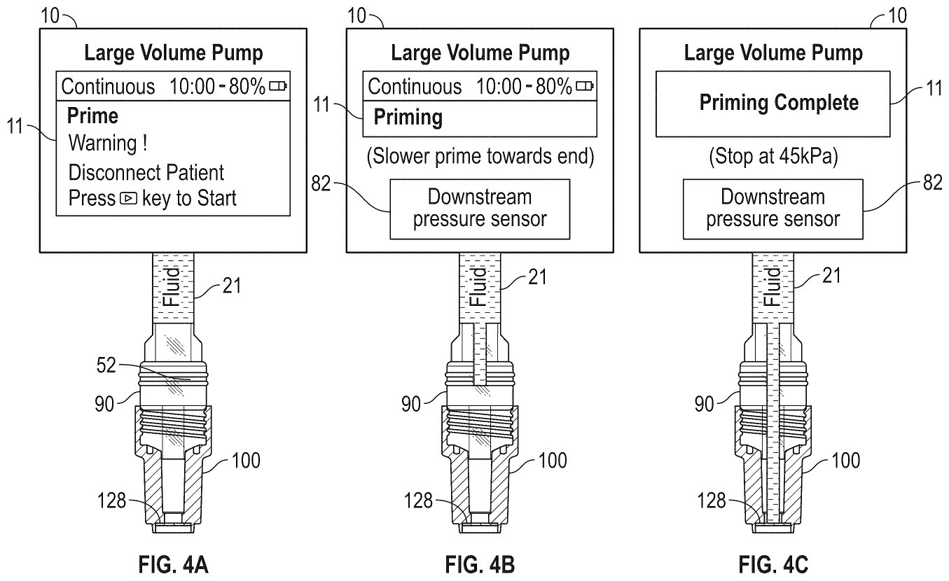

FIGS. 4A, 4B, and 4C depict an example of priming an infusion line using the pressure priming system of the subject technology. In the depicted example, a large volume infusion pump (LVP) 10 initiates priming of a tubing 21 connected to an input of the disclosed pressure priming apparatus 25. In FIG. 4A, the pump 10 is activated and displays a user interface prompting a clinician to begin priming. As shown, the user interface 11 displayed on a display screen associated with pump 10 may provide an alert or warning to disconnect the tubing from the patient and/or downstream intravenous line or catheter. The clinician ensures that the tubing is disconnected from the patient and that the pressure priming apparatus 25 is connected to a distal end of the infusion line, and then activates a control to start the infusion. As will be described further, the pump 10 may be configured to start the priming of the tubing 21 at a first flow rate and then slow the flow rate as the pressure increases.

In the depicted example, the first portion 90 (oriented on top) of the apparatus 25 includes an anti-siphon valve, and the second portion 100 includes a priming cap with a hydrophobic membrane 128 coupled to a luer connector of the first portion 90. At this point, gravity flow of the fluid within the tubing may occur; however, no fluid will pass through the first portion 90 of the apparatus 25 due to a first breakthrough backpressure generated by the first portion.

In FIG. 4B, the pump primes the tubing 21 at a given flow rate until a priming pressure threshold is reached. As described previously, the priming pressure threshold is selected to be greater than a first fluid breakthrough backpressure of the input 52 to the apparatus 25, and less than a second fluid breakthrough backpressure of the output of the apparatus 25. Prior to or during the priming operation, the downstream pressure sensor 82 of the pump 10 is activated to measure the pressure within the tubing 21. The priming operation is initiated, the user interface 11 indicates that the pump is priming, and the pressure in the tubing is allowed to increase over the first breakthrough backpressure so that the fluid begins to enter a chamber within the apparatus 25, as shown. The pressure can be continuously monitored so as to not allow the fluid to overcome the second fluid breakthrough backpressure and spill out of the output 54 of apparatus 25.

According to various implementations, the pump 10 is programmed to prime the fluid tubing 21 at a first flow rate for a first period of time (e.g., before the pressure reaches the selected threshold), and then prime the fluid tubing 21 at a second flow rate until the threshold is satisfied. In some implementations, the pump 10 may initiate a faster prime (e.g., 300 mL/hr) and then slow to a slower prime (e.g., 100 mL/hr) as the downstream sensor 82 measures pressure nearing the selected priming pressure threshold. For example, multiple pressure thresholds may be provided; a first threshold (e.g., equal to or just over the first breakthrough back pressure) for slowing the prime speed, and the (second) priming pressure threshold for terminating the priming operation. In some implementations, the priming pressure threshold may be selected to be half way between the first breakthrough backpressure and the second breakthrough backpressure. If the first breakthrough backpressure is 30 kPa and the second breakthrough backpressure is 60 kPa then the priming pressure threshold may be selected to be 45 kPa.

In some implementations, the flow rate may decrease based on a volume of the chamber within the pressure priming apparatus 25. The pump may delivery a portion of the chamber volume at the higher rate followed by a second or remaining portion at a lower rate. For example, if the chamber volume is 7 mL, the pump 10 may deliver the first 4 mL into the chamber at a high flow rate (e.g., 300 mL/hr) and then switch to the lower flow rate (e.g., 100 mL/hr) for the remaining 3 mL. In some implementations, the rate may continuously slow based on difference between the pressure measured by sensor 82 and the priming pressure threshold.

In FIG. 4C, the downstream pressure sensor 82 of pump 10 detects a pressure satisfying the priming pressure threshold and the priming operation terminates. A notification indicating completion of the priming operation may be displayed on the user interface 11. In the depicted example, the priming operation terminates when the pressure sensor 82 detects a pressure of 45 kPa, which is half way between the first breakthrough backpressure of 45 kPa and the second breakthrough backpressure of 60 kPa. The pump 10 may dynamically identify the priming pressure threshold. For example, the pump 10 may receive information identifying the pressure priming apparatus 25. Based on this information, the pump 10 may determine (e.g., look up table, inquiry service, data store, etc.) a pressure threshold or range for the pressure priming apparatus 25. This ensures that the proper priming is safely performed, in part, by dynamically adjusting the process for the pressure priming apparatus 25. Furthermore, these features ensure that resistance from the pressure priming apparatus 25 does not inadvertently cause occlusion or other pressure related alarms or errors.

Accordingly, priming is automatically accomplished using the pump 10. A clinician does not have to estimate prime volume, or sit and wait, or manually start and stop the pump to top off the fluid in the tubing; the fluid aromatically stops when the pump senses that the fluid reached the correct pressure and is, for example, at the hydrophobic membrane without leakage.

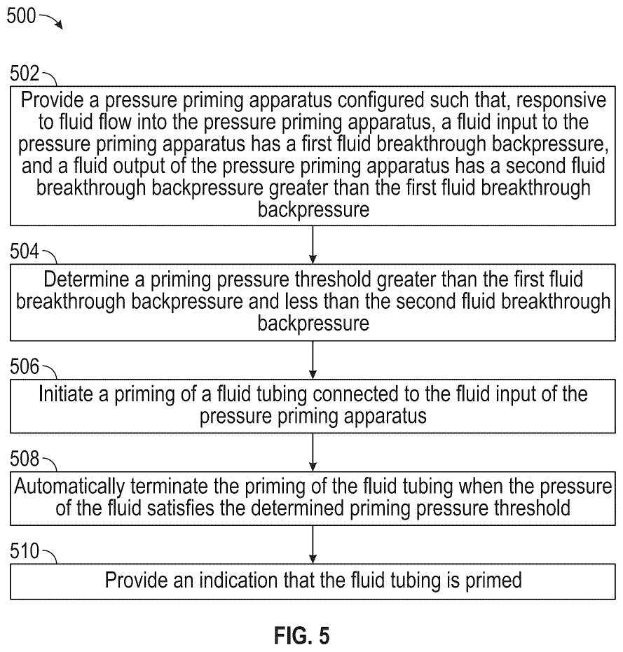

FIG. 5 depicts an example process for automatically priming an infusion line, according to various implementations of the subject technology. For explanatory purposes, the various blocks of example process 500 are described herein with reference to FIGS. 1 through 4, and the components and/or processes described herein. In some implementations, one or more of the blocks may be implemented apart from other blocks, and by one or more different devices. Further for explanatory purposes, the blocks of example process 500 are described as occurring in serial, or linearly. However, multiple blocks of example process 500 may occur in parallel. In addition, the blocks of example process 500 need not be performed in the order shown and/or one or more of the blocks of example process 500 need not be performed.

In the depicted example, a pressure priming apparatus 25 is provided (502). The pressure priming apparatus 25 is configured such that, responsive to fluid flow into the pressure priming apparatus, a fluid input 52 to the pressure priming apparatus has a first fluid breakthrough backpressure, and a fluid output 54 of the pressure priming apparatus has a second fluid breakthrough backpressure greater than the first fluid breakthrough backpressure. In other words, the fluid input 52 may be permeable to fluid at the first fluid breakthrough backpressure, and the fluid output may be permeable to the fluid at the second fluid breakthrough backpressure. According to various implementations, the pressure priming apparatus comprises a first portion 90 and a second portion 100 coupled together. The first portion 90 includes the fluid input 52 and the second portion 100 includes the fluid output 54.

In some implementations, the first portion 90 of the pressure priming apparatus 25 may include a one-way valve at the input to control the directional flow of the fluid. For example, the first portion may include an anti-siphon valve, and the second portion may include a priming cap that couples to a luer fitting of the anti-siphon valve. In this regard, the clinician may utilize an anti-siphon valve and priming cap with predetermined backpressure characteristics known by the pump controller or associated system, as described further below.

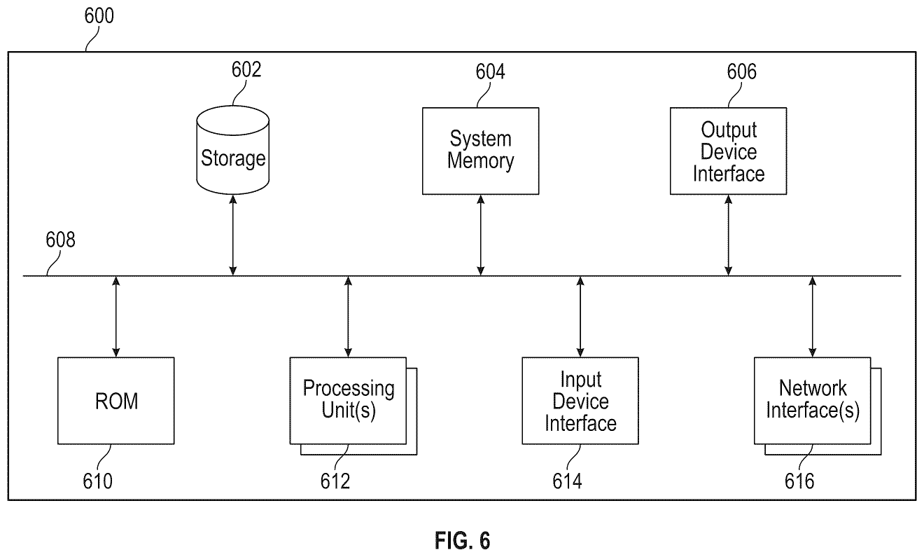

A pump controller (optionally) determines a priming pressure threshold greater than the first fluid breakthrough backpressure and less than the second fluid breakthrough backpressure (504). The pump controller may include a microprocessor of the pump 10, or a control unit or other computing device associated with the pump 10 (see, e.g., FIG. 6). In some implementations, the pump controller determines the priming pressure threshold and/or the backpressures by retrieving their values from a memory (e.g., onboard or offboard memory 604, 610, or over a network connection 616). In this regard, the backpressure characteristics of the first and second portions may be predetermined and stored for use in determining the priming pressure threshold. In some implementations, the priming pressure threshold and/or the backpressures may be manually entered by the clinician via a user interface of the pump 10 or an associated computing device. In some implementations, the priming pressure threshold may be a predetermined value obtained from a memory system. In some implementations, the pump controller may obtain the first and second breakthrough backpressures and then determine the priming pressure threshold based on the backpressures. For example, the pump controller may select a priming pressure threshold halfway between the first fluid breakthrough backpressure and the second fluid breakthrough backpressure.

In some implementations, an identifier associated with the pressure priming apparatus is received by the pump controller or a computing device associated with the pump controller. The identifier may be, for example, scanned from a barcode or electronic identification device affixed or imbedded within the pressure priming apparatus. A clinician may use a scanning device to scan the identifier, and the controller and/or computing device may query a server for the first fluid breakthrough backpressure and the second fluid breakthrough backpressure, which are then obtained from the result.

The pump controller initiates a priming of a fluid tubing connected to the fluid input of the priming pressure apparatus 25 (506). In some implementations, the pump 10 initiates the priming by pumping fluid through the tubing at a predetermined flow rate and allows the pressure to increase beyond the first fluid breakthrough backpressure until the determined priming pressure threshold is reached.

In some implementations, a flow rate of the priming is adjusted (e.g., reduced) according to a differential between the current pressure of the fluid and the priming pressure threshold. In some implementations, the pump controller primes the fluid tubing at a first flow rate (e.g., 300 mL/hr) for a first period of time before a pressure of the fluid reaches the priming pressure threshold, and then primes the fluid tubing at a second flow rate (e.g., 100 mL/hr), lower than the first flow rate, for a second period of time until the pressure satisfies the priming pressure threshold.

The pump controller then automatically terminates the priming of the fluid tubing when the priming pressure of the fluid satisfies the determined priming pressure threshold (508). As described previously, the pump controller may monitor the pressure within the tubing using downstream pressure sensor 82, and may monitor more frequently when the pressure becomes greater than a predetermined threshold. When the pressure reaches the determined priming pressure threshold then the pump controller may terminate the priming operation. According to various implementations, a user interface 11, generated on a display screen associated with the pump 10 or an associated computing device, graphically indicates that the priming is complete when the fluidic pressure of the fluid satisfies the priming pressure threshold (510).

In some implementations, the pump controller initiates the priming of the fluid tubing at a first flow rate for a first period of time before a pressure of the fluid reaches the priming pressure threshold, and determine that the pressure of the fluid has not reached the priming pressure threshold within a predetermined period of time. Responsive to determining that the pressure of the fluid has not reached the priming pressure threshold within the predetermined period of time, a notification may be provided indicating a priming failure. On completion of the priming operation, the clinician may remove the second portion 100 of the pressure priming apparatus from the first portion 90 of the apparatus 25 and connect the infusion set 20 to begin infusion of the fluid to a patient.

In some implementations, a process 550 may include the pump 10 or pump controller, or associated computing device, requesting user confirmation that the second portion is coupled to the first portion, initiating the priming of process 500 responsive to receiving the user confirmation, prompt (e.g., on user interface 11) a user to remove the second portion after the priming is terminated, and cause a pump 10, responsive to receiving an indication that the second portion was removed from the first portion, to pump fluid through the fluid tubing. In some implementations, the pump 10 or pump controller, or associated computing device, may detect activation (e.g., a selection) of a control element (e.g., a menu item) via the user interface 11, and identify the first and/or second portion 90, 100 based on the activation of the control element (e.g., selecting the portion from a menu), or scan information from a portion of the first and/or second portion(s), and decode the information to identify the first and/or second portion(s). In this regard, the priming pressure may be determined based on a lookup of the priming pressure (e.g., in a database) based on the identified first and/or second portion(s).

Many of the above-described example process 500 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROM s, flash drives, RAM chips, hard drives, EPROM s, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 6 is a conceptual diagram illustrating an example electronic system 600 for automatically priming an infusion line, according to aspects of the subject technology. Electronic system 600 may be a computing device for execution of software associated with one or more portions or steps of 500 or components and methods provided by FIGS. 1-5, including but not limited to computing hardware within pump 10 or an associated computing device or associated modules or terminals. Electronic system 600 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and methods previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as, random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM) , a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification

Clause 1. A system for automatically priming an infusion line, comprising: a pressure priming apparatus comprising: a fluid input permeable to fluid at a first fluid breakthrough backpressure; a fluid chamber; and a fluid output permeable to the fluid at a second fluid breakthrough backpressure greater than the first fluid breakthrough backpressure; and a pump controller configured to: determine a priming pressure threshold greater than the first fluid breakthrough backpressure and less than the second fluid breakthrough backpressure; initiate a priming of a fluid tubing connected to the fluid input of the pressure priming apparatus with a fluid; automatically terminate the priming of the fluid tubing when the priming pressure of the fluid satisfies the priming pressure threshold; and provide an indication that the fluid tubing is primed responsive to the fluid satisfying the priming pressure threshold.

Clause 2. The system of Clause 1, wherein the pressure priming apparatus comprises a first portion and a second portion coupled together, the first portion comprising the fluid input, and the second portion comprising the fluid output.

Clause 3. The system of Clause 2, wherein the fluid output is sealed with a hydrophobic membrane, and wherein the second fluid breakthrough backpressure is based on a characteristic of the hydrophobic membrane.

Clause 4. The system of any one of Clauses 1 through 3, where the pump controller is further configured to: request, via a user interface, confirmation that the second portion is coupled to the first portion; initiate the priming responsive to receiving the confirmation; present a prompt, via the user interface, to remove the second portion after the priming is terminated; detect removal of the second portion after the priming is terminated; and cause a pump, responsive to detecting the removal of the second portion, to pump fluid through the fluid tubing.

Clause 5. The system of Clause 4, where the pump controller is further configured to: detect activation of a control element via the user interface, and identify the second portion based on the activation of the control element; or scan information from a portion of the second portion, and decoding the information to identify the second portion, wherein the pump controller being configured to determine the priming pressure comprises the pump controller being configured to look up the priming pressure based on identifying the second portion.

Clause 6. The system of any one of Clauses 1 through 5, where the pump controller is further configured to: receive an identifier associated with the pressure priming apparatus; retrieve, from a data store, the first fluid breakthrough backpressure and the second fluid breakthrough backpressure based on the received identifier; and wherein the pump controller is configured to determine the priming pressure responsive to retrieving the first and second fluid breakthrough backpressures.

Clause 7. The system of any one of Clauses 1 through 6, wherein the pump controller is further configured to: prime the fluid tubing at a first flow rate for a first period of time before a pressure of the fluid reaches the determined priming pressure threshold; and prime the fluid tubing at a second flow rate, lower than the first flow rate, for a second period of time until the pressure satisfies the determined priming pressure threshold.

Clause 8. The system of any one of Clauses 1 through 7, wherein the pump controller is further configured to: reduce a flow rate of the priming according to a differential between a current pressure of the fluid and the determined priming pressure threshold.

Clause 9. The system of any one of Clauses 1 through 8, wherein the pump controller is further configured to: initiate the priming of the fluid tubing at a first flow rate for a first period of time before a pressure of the fluid reaches the determined priming pressure threshold; determine that the pressure of the fluid has not reached the determined priming pressure threshold within a predetermined period of time; and provide a notification indicating a priming failure responsive to determining that the pressure of the fluid has not reached the determined priming pressure threshold within the predetermined period of time.

Clause 10. The system of any one of Clauses 1 through 9, wherein the pump controller is further configured to: graphically indicate on a display screen that the priming is complete when the fluidic pressure of the fluid satisfies the determined priming pressure threshold.

Clause 11. A method for automatically priming an infusion line, comprising: providing a pressure priming apparatus comprising: a fluid input permeable to fluid at a first fluid breakthrough backpressure; a fluid chamber; and a fluid output permeable to the fluid at a second fluid breakthrough backpressure greater than the first fluid breakthrough backpressure; determine a priming pressure threshold greater than the first fluid breakthrough backpressure and less than the second fluid breakthrough backpressure; initiating a priming of a fluid tubing connected to the fluid input of the pressure priming apparatus with a fluid; automatically terminating the priming of the fluid tubing when the priming pressure of the fluid satisfies the determined priming pressure threshold; and providing an indication that the fluid tubing is primed responsive to the fluid satisfying the determined priming pressure threshold.

Clause 12. The method of Clause 11, wherein the pressure priming apparatus comprises a first portion and a second portion coupled together, the first portion comprising the fluid input, and the second portion comprising the fluid output, the method further comprising: requesting user confirmation that the second portion is coupled to the first portion; initiating the priming responsive to receiving the user confirmation; prompting a user to remove the second portion after the priming is terminated; and causing a pump, responsive to receiving an indication that the second portion was removed from the first portion, to pump fluid through the fluid tubing.

Clause 13. The method of Claim 12, the method further comprising: detecting activation of a control element via the user interface, and identifying the second portion based on the activation of the control element; or scanning information from a portion of the second portion, and decoding the information to identify the second portion, wherein determining the priming pressure comprises looking up the priming pressure based on identifying the second portion.

Clause 14. The method of any one of Clauses 11 through 13, further comprising: receiving an identifier associated with the pressure priming apparatus; determining the first fluid breakthrough backpressure and the second fluid breakthrough backpressure based on the received identifier; and wherein the pump controller is configured to determine the priming pressure responsive to retrieving the first and second fluid breakthrough backpressures.

Clause 15. The method of any one of Clauses 11 through 14, further comprising: priming the fluid tubing at a first flow rate for a first period of time before a pressure of the fluid reaches the determined priming pressure threshold; and priming the fluid tubing at a second flow rate, lower than the first flow rate, for a second period of time until the pressure satisfies the determined priming pressure threshold.

Clause 16. The method of any one of Clauses 11 through 15, further comprising: reduce a flow rate of the priming according to a differential between a current pressure of the fluid and the determined priming pressure threshold.

Clause 17. The method of any one of Clauses 11 through 16, further comprising: initiating the priming of the fluid tubing at a first flow rate for a first period of time before a pressure of the fluid reaches the determined priming pressure threshold; determining that the pressure of the fluid has not reached the determined priming pressure threshold within a predetermined period of time; and providing a notification indicating a priming failure responsive to determining that the pressure of the fluid has not reached the determined priming pressure threshold within the predetermined period of time.

Clause 18. The method of any one of Clauses 11 through 17, further comprising: graphically indicate on a display screen that the priming is complete when the fluidic pressure of the fluid satisfies the determined priming pressure threshold.

Clause 19. The method of any one of Clauses 11 through 18, wherein providing the pressure priming apparatus comprises: coupling a first portion of the pressure priming apparatus and a second portion of the pressure priming apparatus together, the first portion comprising the fluid input, and the second portion comprising the fluid output.

Clause 20. A non-transitory computer readable medium having instructions stored thereon that, when executed, perform operations comprising: identifying a pressure priming apparatus comprising: a fluid input permeable to fluid at a first fluid breakthrough backpressure; and a fluid output permeable to the fluid at a second fluid breakthrough backpressure greater than the first fluid breakthrough backpressure; determine a priming pressure threshold greater than the first fluid breakthrough backpressure and less than the second fluid breakthrough backpressure; initiating a priming of a fluid tubing connected to the fluid input of the pressure priming apparatus with a fluid; automatically terminating the priming of the fluid tubing when the priming pressure of the fluid satisfies the determined priming pressure threshold; and providing an indication that the fluid tubing is primed responsive to the fluid satisfying the determined priming pressure threshold.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A system for automatically priming an infusion line, comprising:
a pressure priming apparatus comprising:
a fluid input permeable to fluid at a first fluid breakthrough backpressure;
a fluid chamber; and
a fluid output permeable to the fluid at a second fluid breakthrough backpressure greater than the first fluid breakthrough backpressure; and
a pump controller configured to:
determine a priming pressure threshold greater than the first fluid breakthrough backpressure and less than the second fluid breakthrough backpressure;
initiate a priming of a fluid tubing connected to the fluid input of the pressure priming apparatus with a fluid;
automatically terminate the priming of the fluid tubing when the priming pressure of the fluid satisfies the priming pressure threshold; and
provide an indication that the fluid tubing is primed responsive to the fluid satisfying the priming pressure threshold.

2. The system of claim 1, wherein the pressure priming apparatus comprises a first portion and a second portion coupled together, the first portion comprising the fluid input, and the second portion comprising the fluid output.

3. The system of claim 2, wherein the fluid output is sealed with a hydrophobic membrane, and wherein the second fluid breakthrough backpressure is based on a characteristic of the hydrophobic membrane.

4. The system of claim 2, where the pump controller is further configured to:
request, via a user interface, confirmation that the second portion is coupled to the first portion;
initiate the priming responsive to receiving the confirmation;
present a prompt, via the user interface, to remove the second portion after the priming is terminated;
detect removal of the second portion after the priming is terminated; and
cause a pump, responsive to detecting the removal of the second portion, to pump fluid through the fluid tubing.

5. The system of claim 2, where the pump controller is further configured to:
detect activation of a control element via the user interface, and identify the second portion based on the activation of the control element; or
scan information from a portion of the second portion, and decoding the information to identify the second portion,
wherein the pump controller being configured to determine the priming pressure comprises the pump controller being configured to look up the priming pressure based on identifying the second portion.

6. The system of claim 1, where the pump controller is further configured to:
receive an identifier associated with the pressure priming apparatus;
retrieve, from a data store, the first fluid breakthrough backpressure and the second fluid breakthrough backpressure based on the received identifier; and
wherein the pump controller is configured to determine the priming pressure responsive to retrieving the first and second fluid breakthrough backpressures.

7. The system of claim 1, wherein the pump controller is further configured to:
prime the fluid tubing at a first flow rate for a first period of time before a pressure of the fluid reaches the determined priming pressure threshold; and
prime the fluid tubing at a second flow rate, lower than the first flow rate, for a second period of time until the pressure satisfies the determined priming pressure threshold.

8. The system of claim 1, wherein the pump controller is further configured to:
reduce a flow rate of the priming according to a differential between a current pressure of the fluid and the determined priming pressure threshold.

9. The system of claim 1, wherein the pump controller is further configured to:
initiate the priming of the fluid tubing at a first flow rate for a first period of time before a pressure of the fluid reaches the determined priming pressure threshold;
determine that the pressure of the fluid has not reached the determined priming pressure threshold within a predetermined period of time; and
provide a notification indicating a priming failure responsive to determining that the pressure of the fluid has not reached the determined priming pressure threshold within the predetermined period of time.

10. The system of claim 1, wherein the pump controller is further configured to:
graphically indicate on a display screen that the priming is complete when the fluidic pressure of the fluid satisfies the determined priming pressure threshold.

11. A method for automatically priming an infusion line, comprising:
providing a pressure priming apparatus comprising:
a fluid input permeable to fluid at a first fluid breakthrough backpressure;
a fluid chamber; and
a fluid output permeable to the fluid at a second fluid breakthrough backpressure greater than the first fluid breakthrough backpressure;
determine a priming pressure threshold greater than the first fluid breakthrough backpressure and less than the second fluid breakthrough backpressure;
initiating a priming of a fluid tubing connected to the fluid input of the pressure priming apparatus with a fluid;
automatically terminating the priming of the fluid tubing when the priming pressure of the fluid satisfies the determined priming pressure threshold; and
providing an indication that the fluid tubing is primed responsive to the fluid satisfying the determined priming pressure threshold.

12. The method of claim 11, wherein the pressure priming apparatus comprises a first portion and a second portion coupled together, the first portion comprising the fluid input, and the second portion comprising the fluid output, the method further comprising:
requesting user confirmation that the second portion is coupled to the first portion;
initiating the priming responsive to receiving the user confirmation;
prompting a user to remove the second portion after the priming is terminated; and
causing a pump, responsive to receiving an indication that the second portion was removed from the first portion, to pump fluid through the fluid tubing.

13. The method of claim 12, the method further comprising:
detecting activation of a control element via the user interface, and identifying the second portion based on the activation of the control element; or
scanning information from a portion of the second portion, and decoding the information to identify the second portion,
wherein determining the priming pressure comprises looking up the priming pressure based on identifying the second portion.

14. The method of claim 11, further comprising:
receiving an identifier associated with the pressure priming apparatus;
determining the first fluid breakthrough backpressure and the second fluid breakthrough backpressure based on the received identifier; and
wherein the pump controller is configured to determine the priming pressure responsive to retrieving the first and second fluid breakthrough backpressures.

15. The method of claim 11, further comprising:
priming the fluid tubing at a first flow rate for a first period of time before a pressure of the fluid reaches the determined priming pressure threshold; and
priming the fluid tubing at a second flow rate, lower than the first flow rate, for a second period of time until the pressure satisfies the determined priming pressure threshold.

16. The method of claim 1, further comprising:
reduce a flow rate of the priming according to a differential between a current pressure of the fluid and the determined priming pressure threshold.

17. The method of claim 11, further comprising:
initiating the priming of the fluid tubing at a first flow rate for a first period of time before a pressure of the fluid reaches the determined priming pressure threshold;
determining that the pressure of the fluid has not reached the determined priming pressure threshold within a predetermined period of time; and
providing a notification indicating a priming failure responsive to determining that the pressure of the fluid has not reached the determined priming pressure threshold within the predetermined period of time.

18. The method of claim 11, further comprising:
graphically indicate on a display screen that the priming is complete when the fluidic pressure of the fluid satisfies the determined priming pressure threshold.

19. The method of claim 11, wherein providing the pressure priming apparatus comprises:
coupling a first portion of the pressure priming apparatus and a second portion of the pressure priming apparatus together, the first portion comprising the fluid input, and the second portion comprising the fluid output.

20. A non-transitory computer readable medium having instructions stored thereon that, when executed, perform operations comprising:
identifying a pressure priming apparatus comprising:
a fluid input permeable to fluid at a first fluid breakthrough backpressure; and
a fluid output permeable to the fluid at a second fluid breakthrough backpressure greater than the first fluid breakthrough backpressure;
determine a priming pressure threshold greater than the first fluid breakthrough backpressure and less than the second fluid breakthrough backpressure;
initiating a priming of a fluid tubing connected to the fluid input of the pressure priming apparatus with a fluid;
automatically terminating the priming of the fluid tubing when the priming pressure of the fluid satisfies the determined priming pressure threshold; and
providing an indication that the fluid tubing is primed responsive to the fluid satisfying the determined priming pressure threshold.
